(19) United States
(12) Patent Application Publication
(10) Pub. No.: US 2021/0318302 A1
(43) Pub. Date: Oct. 14, 2021

(54) AMINO ACID-MODIFIED NANOPORES AND USES THEREOF

(71) Applicant: Yissum Research Development Company of the Hebrew University of Jerusalem Ltd., Jerusalem (IL)

(72) Inventors: Danny Porath, Jerusalem (IL); Meital Reches, Bet Hasmonai (IL); Dvir Marom Rotem, Jerusalem (IL); Abeer Karmi, East Jerusalem (IL)

(21) Appl. No.: 16/766,999

(22) PCT Filed: Nov. 27, 2018
(86) PCT No.: PCT/IL2018/051294

§ 371 Date: May 26, 2020

Related U.S. Application Data
(60) Provisional application No. 62/590,766, filed on Nov. 27, 2017.

(51) Int. Cl. G01N 33/543; G01N 33/487

(52) U.S. Cl. CPC G01N 33/54393; G01N 27/44756; G01N 33/48721

(57) ABSTRACT

The invention provides a nanopore assembly and a device comprising same, wherein the nanopores assembly is amino acid modified to endow the assembly with selected properties.

### TECHNOLOGICAL FIELD

The present disclosure relates to amino acid-modified nanopores and uses thereof.

### BACKGROUND

Nanopores, pores of nanometer dimensions in an electrically insulating membrane, have shown promise for use in a variety of sensing applications, including single molecule detectors. Solid-state nanopores are generally made in silicon compound membranes, one of the most common being silicon nitride.

To influence the properties of the nanopore, investigators have associated the nanopore surface with various materials.

Derivatization of silicon nitride nanopores with nitrilotriacetic acid receptors was reported for the stochastic sensing of proteins [1].

Kowalczyk et al [2] reported selective transport of proteins across individual biomimetic nuclear pore complexes at the single-molecule level.

Liebes-Peer et al [3] demonstrate the use of de novo designed peptides for functionalization of nanopores that enable the detection of small analytes at the single molecule level. The detection relies on a cooperative peptide conformational change that is induced by the binding of the small molecule to a receptor domain on the peptide.

International patent application [4] discloses a hybrid structure comprising nanopores and ring-like polypeptides that are situated at the vicinity of the nanopore. The hybrid structure may be used in a variety of diagnostic and synthetic applications.

### REFERENCES

[1] Ruoshan Wei et al., Stochastic sensing of proteins with receptor-modified solid-state nanopores, Nature Nanotechnology, 7, 257-263 (2012);
[2] Stefan W. Kowalczyk et al., Single-molecule transport across an individual biomimetic nuclear pore complex, Nature Nanotechnology, 6, 433-438 (2011);
[3] Yael Liebes-Peer, et al., Amplification of Single Molecule Translocation Signal Using β-Strand Peptide Functionalized Nanopores, ACS Nano, 2014, 7, 6822-6832;
[4] WO 2014122654.

### SUMMARY OF THE INVENTION

The methodology that is at the core of the technology disclosed herein is the ability to modify a nanopore structure with amino acids or derivatives thereof. The modification occurs through a self-assembly of at least one amino acid or a derivative thereof that contains surface-associating group(s), such as DOPA, and optionally at least one other group or residue that endows the modified nanopore with new functionalities (e.g. charge, hydrophilicity). Modifying the nanopores with different amino acids or derivatives thereof allows tailoring nanopores to adopt certain predefined new characteristics and thus various uses or applications.

The modified nanopores (i.e., nanopores associated with an amino acid or a derivative thereof, as disclosed) may be utilized in detection of biopolymers such as DNA, proteins, nanoparticles, clusters of nanoparticles and localized pH and salt sensing. The amino acids or derivatives thereof also contribute to the stability of the nanopores. As demonstrated herein, the usability period, as compared to untreated nanopores, is increased dramatically, e.g., from one day to several months.

The inventors have further found that the translocation rate (time) of analytes through the modified nanopore was slower as compared to the translocation through bare synthetic nanopores.

Nanopore-based sensors allow analysis of various materials (such as metal ions, small molecules, reactive molecules, proteins and DNA), at a single molecule level with sub-nanometer resolution and without needing to resort to expensive labels or error-inclined amplifications. The method disclosed herein is based on maintaining a constant flow of ions, in solution, through a nano-sized hole in a membrane. When a single molecule under investigation partially blocks the pore, a change in the ion flow is detected and measured electrically, indicating a typical blocking level of a section of the molecule inside the hole. The most apparent application of this technology is rapid and low cost DNA sequencing by translocating DNA through the nanopore. The measured ion flow changes during translocation of the DNA molecule through the nanopore that can be translated to the sequence of bases in the measured DNA.

Additional applications and uses may include detection of various analytes based on their charge, size, structure and other variables, controlling the dynamics of translocated biopolymer (such as DNA, RNA and proteins) transport through the nanopore in order to ease their detection and sequencing and others.

As will be demonstrated herein, the amino acid-coating contributes to the stability of the nanopores e.g. the time that these nanopores can be used without further treatment; which compared to untreated nanopores, is increased dramatically (from one day to at least few months).

Thus, in accordance with a first aspect of the invention, there is provided a nanopore or a nanopore assembly comprising two or more such nanopores, each nanopore being surface associated with at least one amino acid or a derivative thereof, the association being between the amino acid or derivative thereof and at least one of (i) an outer rim surface region of the nanopore, (ii) an inner-pore region of the nanopore, and/or (iii) a circumference surface of the nanopore rim, wherein optionally the amino acid is 3,4-dihydroxyphenylalanine (DOPA) and the amino acid derivative is optionally a DOPA-containing molecule.

In some embodiments, the amino acid is DOPA.

In some embodiments, the amino acid is not DOPA, but is selected amongst amino acids capable of surface-association.

As noted, alternatively to modifying the nanopore with an amino acid, as defined, the nanopore may be modified with an “amino acid derivative”, being an amino acid substituted with an atom or a group of atoms. The amino acid derivative may be of the form AA-X, wherein AA is an amino acid or a peptide, as defined herein, X is a substituting group, and designates a covalent bond. The amino acid derivative may be substituted with one or more such X groups.

In some embodiments, group X is selected from hydrophobic groups, hydrophilic groups, electron withdrawing groups, electron donating groups, bulky groups, single atom substituents, binary substituents and others.

In some embodiments, X is selected from H, —C₁-C₂₀alkyl, —C₂-C₂₀alkenyl, —C₂-C₂₀alkynyl, —(O—(CH₂)n)-, —(C═O)—C₁-C₂₀alkyl, —(C═O)—C₂-C₂₀alkenyl, —(C═O)—C₂-C₂₀alkynyl, —(C═O)—O—C₁-C₂₀alkyl, —(C═O)—O—C₂-C₂₀alkenyl, —(C═O)—O—C₂-C₂₀alkynyl, —O—(C═O)—C₁-C₂₀alkyl, —O—(C═O)—C₂-C₂₀alkenyl, —O—(C═O)—C₂-C₂₀alkynyl, —(C═O)—NR—C₁-C₂₀alkyl, —(C═O)—NR—C₂-C₂₀alkenyl, —(C═O)—NR—C₂-C₂₀alkynyl, —NR—(C═O)—C₁-C₂₀alkyl, —NR—(C═O)—C₂-C₂₀alkenyl, —NR—(C═O)—C₂-C₂₀alkynyl, —(C═O)—C₁-C₂₀alkylene-NRR′—, —(C═O)—C₂-C₂₀alkenylene-NRR′—, —(C═O)—C₂-C₂₀alkynylene-NRR′—, amino acid, a peptide such as a dipeptide, a tripeptide, a tetrapeptide or longer peptides, and a nucleic acid. In the above, each R or R′, independently of another, is H, —C₁-C₂₀alkyl, —C₂-C₂₀alkenyl, —C₂-C₂₀alkynyl, —(O—(CH₂)n)-, —(C═O)—C₁-C₂₀alkyl, —(C═O)—C₂-C₂₀alkenyl, —(C═O)—C₂-C₂₀alkynyl, —(C═O)—O—C₁-C₂₀alkyl, —(C═O)—O—C₂-C₂₀alkenyl, —(C═O)—O—C₂-C₂₀alkynyl, —O—(C═O)—C₁-C₂₀alkyl, —O—(C═O)—C₂-C₂₀alkenyl or —O—(C═O)—C₂-C₂₀alkynyl.

In some embodiments, X is selected from H, —C₁-C₂₀alkyl, —C₂-C₂₀alkenyl, —C₂—C, —(O—(CH₂)n)-, —(C═O)—C₁-C₂₀alkyl, —(C═O)—C₂-C₂₀alkenyl, —(C═O)—C₂-C₂₀alkynyl, —(C═O)—O—C₁-C₂₀alkyl, —(C═O)—O—C₂-C₂₀alkenyl, —C═O)—O—C₂-C₂₀alkynyl, —O—(C═O)—C₁-C₂₀alkyl, —O—(C═O)—C₂-C₂₀alkenyl, —O—(C═O)—C₂-C₂₀alkynyl, —(C═O)—NR—C₁-C₂₀alkyl, —(C═O)—NR—C₂-C₂₀alkenyl, —(C═O)—NR—C₂-C₂₀alkynyl, —NR—(C═O)—C₁-C₂₀alkyl, —NR—(C═O)—C₂-C₂₀alkenyl, —NR—(C═O)—C₂-C₂₀alkynyl, —(C═O)—C₁-C₂₀alkylene-NRR′—, —(C═O)—C₂-C₂₀alkenylene-NRR′—, —(C═O)—C₂-C₂₀alkynylene-NRR′—, amino acid and a peptide, wherein each R or R′, independently of another, is H, —C₁-C₂₀alkyl, —C₂-C₂₀alkenyl, —C₂-C₂₀alkynyl, —(O—(CH₂)n)-, —(C═O)—C₁-C₂₀alkyl, —(C═O)—C₂-C₂₀alkenyl, —(C═O)—C₂-C₂₀alkynyl, —(C═O)—O—C₁-C₂₀alkyl, —(C═O)—O—C₂-C₂₀alkenyl, —(C═O)—O—C₂-C₂₀alkynyl, —O—(C═O)—C₁-C₂₀alkyl, —O—(C═O)—C₂-C₂₀alkenyl or —O—(C═O)—C₂-C₂₀alkynyl and wherein each alkyl, alkenyl, alkynyl, alkylene, alkenylene and alkynylene is optionally substituted by one or more substituents selected from a nitro group, a hydroxyl group, a mercapto group, a cyano group, an amine, a halide, a sulfo group, a sulfoxide group, a C₁-C₅alkyl, a C₂-C₅alkenyl, a C₂-C₅alkynyl, a C₅-C₁₀cycloalkyl, a C₅-C₁₀cycloalkenyl, a C₅-C₁₀cycloalkynyl, a C₆-C₁₀aryl, a C₅-C₁₀heteroaryl and a C₅-C₁₀heterocyclyl.

In some embodiments, X is a fluorine atom or a fluorine containing group. In some embodiments, the fluorine containing group is a fluorinated alkyl. In some embodiments, the alkyl is perfluorinated.

The variant substituting group X may be covalently associated with the amino acid N-terminal (namely through the amine nitrogen atom) and/or the amino acid C terminal (namely through the amino acid carboxyl end) and/or the alpha-carbon or a side chain. For example, where the amino acid is of the general structure H₂N—CHR—COOH, the amino acid derivative may have the structure H₂N—CHR—COOX, H₂N—CHX—COOH, H₂N—CXR—COOH, HXN—CHR—COOH, X₂N—CHR—COOH, H₂N—CXR—COOX, H₂N—CHX—COOX, X₂N—CHR—COOX, HXN—CXR—COOH, and other similar derivatives, wherein each X may be the same or different.

In some embodiments, the number of X groups may be 0 or 1 or 2 or 3 or 4 or 5 or 6 or 7 or 8 or 9 or 20.

As used herein, -“alkyl”, “alkenyl” and “alkynyl” groups are carbon chains each containing a number of carbon atoms, as indicated, and no double or triple bonds, or at least one double, or at least one triple bond, respectively. Alkenyl carbon chains may contain 1 to 8 double bonds, or 1 to 7 double bonds, or 1 to 6 double bonds, or 1 to 5 double bonds, or 1 to 4 double bonds, or 1 to 3 double bonds, or 1 double bond, or 2 double bonds. Alkynyl carbon chains may similarly contain 1 to 8 triple bonds, or 1 to 7 triple bonds, or 1 to 6 triple bonds, or 1 to 5 triple bonds, or 1 to 4 triple bonds, or 1 to 3 triple bonds, or 1 triple bond, or 2 triple bonds. Exemplary alkyl, alkenyl and alkynyl groups include, but are not limited to, methyl, ethyl, propyl, isopropyl, isobutyl, n-butyl, sec-butyl, tert-butyl, isohexyl, allyl (propenyl) and propargyl (propynyl).
“cycloalkyl” refers to a saturated mono- or multi-cyclic ring system, containing the indicated number of carbon atoms; cycloalkenyl and cycloalkynyl refer to mono- or multicyclic ring systems that respectively include at least one double bond and at least one triple bond. The ring systems of the cycloalkyl, cycloalkenyl and cycloalkynyl groups may be composed of one ring or two or more rings which may be joined together in a fused, bridged or spiro-connected fashion.
“aryl” refers to aromatic monocyclic or multicyclic groups containing the indicated number of carbon atoms. Aryl groups include, but are not limited to groups such as unsubstituted or substituted fluorenyl, unsubstituted or substituted phenyl, benzyl and unsubstituted or substituted naphthyl.
“heteroaryl” refers to a monocyclic or multicyclic aromatic ring system, wherein 1 to 3 of the atoms in the ring system is a heteroatom, that is, an element other than carbon, including e.g., nitrogen, oxygen or sulfur. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl.
“—NRR′” refers to an amine group wherein R and R′ are independently selected as disclosed or from hydrogen, alkyl, alkenyl, alkenyl, cycloalkyl, cycloalkenyl, cycloalkynyl, aryl, heteroaryl, heterocyclyl, ester and carbonyl, each as defined herein or alternatively known in the art.

In some embodiments, in an amino acid derivative of the structure AA-X, X may be as selected above and the amino acid AA may be DOPA. In such embodiments, the amino acid derivative is of the structure DOPA-X, wherein X is a variant group covalently associated as defined.

In some embodiments, X is an amino acid and DOPA-X is a peptide.

The nanopore of the invention is present in a solid substrate i.e. a solid substrate having at least one nanopore perforating therethrough. In some embodiments, the at least one amino acid or a derivative thereof is associated with a region at the top surface of the substrate near or at the nanopore region, at the bottom surface of the substrate near or at the nanopore region, at the opening of the nanopore, and/or at an interior surface region of the nanopore. Amino acid-modified region(s) of the substrate is/are at the vicinity of the nanopore or in the nanopore itself, such that the presence of the amino acid or derivative thereof imposes or endows the nanopores with the desired characteristics.

The nanopores of the invention are referred to herein as ‘modified nanopores’, ‘amino-acid modified nanopore’, ‘peptide-modified nanopores’, ‘nanopore structure’ or simply ‘nanopores’. These terms are used interchangeably to denote nano-holes or nano-sized openings in a solid substrate, which render the substrate three-dimensionally perforated. The substrate having two faces, one of the nanopore openings is present at a first face of the substrate and the second opening is present at a second opposite face of the substrate. Each nanopore is thus a channel that extends the thickness of the substrate. The two openings are connected by an interior defined by the height (length, depth) of the pore. The interior is an open interior or channel allowing flow throughout of any medium, e.g., a liquid medium or any material.

The first opening and the second opening of the nanopore are each characterized by a diameter that may be similar or different. When referring to opposite faces of the nanopore, it is noted that the two openings may be considered essentially parallel or nearly parallel. In some embodiments, the two openings are co-axially positioned.

The nanopore has, on average, a diameter of up to about 50 nm. In some embodiments, the diameter is between about 1 nm and about 50 nm. In further embodiments, the diameter is between about 1 nm and about 20 nm, between about 2 nm and about 10 nm, between about 3 nm and about 8 nm, or between about 3 nm and 5 nm.

In some embodiments, the interior of the nanopore spanning the first opening and the second opening has a length from about 5 nm to about 50 nm; in some other embodiments, from about 10 nm to about 40 nm; in some further embodiments, from about 20 nm to about 35 nm. The nanopore interior length may or may not be identical to the thickness of the substrate in which the pores are provided.

As known in the art, the nanopores may be formed by ‘drilling’ the nanopores in a solid substrate or alternatively by manufacturing a substrate material that is decorated with one or more or a plurality of pores. For example, fabrication of nanopore(s) within a solid substrate may be achieved by any one or more of the following non-limiting processes: feedback controlled low energy (0.5-5.0 keV) gas (e.g., gallium, helium, neon) ion beam sculpting, focused ion beam (based on gallium, helium and neon (1-50 keV)) and high-energy (200-300 keV) electron beam illumination. The nanopore properties, such as for example diameter and length, may be determined by known methods in the field, such as transmission electron microscopy (TEM) and/or atomic force microscopy (AFM).

The solid substrate may comprise a plurality of nanopores, namely an assembly or collection of nanopores. The plurality of nanopores may be arranged in an array of nanopores, wherein in the array the nanopores are in groups or in a pattern, wherein each group or pattern of nanopores is homogeneous or heterogeneous in at least one parameter selected from nanopore density, nanopore size, nanopore depth and nanopore structure. The nanopores may similarly be the same or different in the amino acid-based material they are associated with. As a person versed in the art would appreciate, for certain applications, one group of nanopores may have on average the same pore diameter, while another group of nanopores is formed to have a different pore diameter. In other cases, each group of nanopores may be formed to comprise a plurality of nanopores having different pore diameter.

In accordance with the present disclosure, the “solid substrate” is a solid continuous material in which one or more nanopores are situated. The thickness of the substrate may define the length or depth of the nanopore channel, provided that the substrate is flat or at least homogenous in thickness. Where the substrate is decorated with cavities or is not fully flat, the thickness of the substrate may not be an indication of or may not define the length or depth of the nanopore channel.

The solid substrate is characterized by having a first face or surface and an opposite face or a second face or surface. The distance between the first and second faces may thus, as explained above, define the thickness of the substrate and the length or depth of the nanopore structure. In other words, when referring to the first face and second face of the substrate, it should be referred to planar surfaces of the substrate that are the faces (top end and/or bottom) of the substrate. In some embodiments, the first surface or face and the second surface or face are substantially parallel to each other.

Once the nanopores are perforated through the substrate, from one face to the other, the substrate may be referred to as a membrane. When referring to the solid membrane, it should be noted that it does not encompass a cellular membrane or a bi-lipid layer membrane. In some embodiments, the solid substrate is synthetic. In some other embodiments, the solid substrate is an inorganic sheet, being optionally of at least one metal. In some embodiments, the solid substrate comprises a material selected from silicon, aluminum, titanium, hafnium, graphene, glass, quartz, diamond, gold and teflon.

In some embodiments, the solid substrate is comprised of or is a doped material, such as doped silicon or doped diamond or any of the materials listed above in doped forms.

In some other embodiments, the solid substrate is comprised of or is of an undoped material, as defined herein.

In some embodiments, the solid substrate is selected of a material comprising at least one of silicon nitride (SiN), silicon dioxide (SiO₂), aluminum oxide (Al₂O₃), titanium oxide (TiO₂) hafnium oxide (HfO₂) and graphene.

In some embodiments, the solid substrate consists or comprises silicon nitride (SiN).

At least one pore in a given surface perforated with nanopores, is a nanopore according to the invention, namely a nanopore that is associated with, or modified with, or decorated with, or incorporated with, or comprised of at least one surface-associated or surface-adsorbed amino acid or a derivative thereof. As noted hereinabove, in some embodiments, the amino acid derivative, being of the structure AA-X, as defined, may be a peptide. Generally speaking, a peptide may comprise two or more amino acid residues, connected by peptide bonds. The amino acid, as used herein, in reference to an amino-acid-modified nanopore, and/or in reference to an amino acid derivative, and/or in reference to an amino acid making up a peptide, is a naturally occurring or synthetic amino acid, an amino acid analog, or an amino acid mimetic that functions in a manner similar to a naturally occurring amino acid.

Naturally occurring amino acids are those encoded by the genetic code, as well as those amino acids that are later modified, e.g., hydroxyproline, γ-carboxyglutamate, and O-phosphoserine. Amino acid analogs are compounds that have the same fundamental chemical structure as naturally occurring amino acids, i.e., alpha carbon that is bound to a hydrogen, a carboxyl group, an amino group, and an R group, e.g., homoserine, norleucine, methionine sulfoxide, methionine methyl sulfonium. Such analogs have modified R groups or modified peptide backbones, but retain the same basic chemical structure as a naturally occurring amino acid. Amino acid mimetics are chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

The amino acid (AA) used to modify a nanopore or the at least one amino acid in a peptide used according to the invention is selected amongst amino acids capable of surface association. Such amino acids may be natural, synthetic or semi-synthetic. One such example is 3,4-dihydroxyphenylalanine (DOPA). The other amino acids may be any one or more amino acids selected as herein. The other amino acids may be the same or all different or comprise a combination or a mix of different amino acids.

The amino acids may be selected on the basis of their polarity, charge, solubility, hydrophobicity, hydrophilicity, and/or the amphiphathic nature, nonpolar “hydrophobic” amino acids may be selected amongst valine, isoleucine, leucine, methionine, phenylalanine, tryptophan, cysteine, alanine, tyrosine, histidine, threonine, serine, proline, glycine, arginine and lysine; “polar” amino acids may be selected from arginine, lysine, aspartic acid, glutamic acid, asparagine, glutamine; “positively charged” amino acids may be selected form arginine, lysine and histidine and “acidic” amino acids may be selected from aspartic acid, asparagine, glutamic acid and glutamine.

In some embodiments, the amino acid is selected amongst alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine valine, pyrrolysine and selnocysteine; and amino acid analogs such as homo-amino acids, N-alkyl amino acids, dehydroamino acids, aromatic amino acids and α,α-disubstituted amino acids, e.g., cystine, 5-hydroxylysine, 4-hydroxyproline, α-aminoadipic acid, α-amino-n-butyric acid, 3,4-dihydroxyphenylalanine, homoserine, α-methylserine, ornithine, pipecolic acid, ortho, meta or para-aminobenzoic acid, citrulline, canavanine, norleucine, d-glutamic acid, aminobutyric acid, L-fluorenylalanine, L-3-benzothienylalanine and thyroxine.

In some embodiments, the amino acid is selected amongst aromatic amino acids. Non-limiting examples of aromatic amino acids include tryptophan, tyrosine, naphthylalanine, and phenylalanine. In some embodiments, the amino acid is phenylalanine or derivatives thereof.

In some embodiments, the phenylalanine derivatives is 4-methoxy-phenylalanine, 4-carbamimidoyl-1-phenylalanine, 4-chloro-phenylalanine, 3-cyano-phenylalanine, 4-bromo-phenylalanine, 4-cyano-phenylalanine, 4-hydroxymethyl-phenylalanine, 4-methyl-phenylalanine, 1-naphthyl-alanine, 3-(9-anthryl)-alanine, 3-methyl-phenylalanine, m-amidinophenyl-3-alanine, phenylserine, benzylcysteine, 4,4-biphenylalanine, 2-cyano-phenylalanine, 2,4-dichloro-phenylalanine, 3,4-dichloro-phenylalanine, 2-chloro-penylalanine, 3,4-dihydroxy-phenylalanine, 3,5-dibromotyrosine, 3,3-diphenylalanine, 3-ethyl-phenylalanine, 3,4-difluoro-phenylalanine, 3-chloro-phenylalanine, 3-chloro-phenylalanine, 2-fluoro-phenylalanine, 3-fluoro-phenylalanine, 4-amino-L-phenylalanine, homophenylalanine, 3-(8-hydroxyquinolin-3-yl)-1-alanine, 3-iodo-tyrosine, kynurenine, 3,4-dimethyl-phenylalanine, 2-methyl-phenylalanine, m-tyrosine, 2-naphthyl-alanine, 5-hydroxy-1-naphthalene, 6-hydroxy-2-naphthalene, meta-nitro-tyrosine, (beta)-beta-hydroxy-1-tyrosine, (beta)-3-chloro-beta-hydroxy-1-tyrosine, o-tyrosine, 4-benzoyl-phenylalanine, 3-(2-pyridyl)-alanine, 3-(3-pyridyl)-alanine, 3-(4-pyridyl)-alanine, 3-(2-quinolyl)-alanine, 3-(3-quinolyl)-alanine, 3-(4-quinolyl)-alanine, 3-(5-quinolyl)-alanine, 3-(6-quinolyl)-alanine, 3-(2-quinoxalyl)-alanine, styrylalanine, pentafluoro-phenylalanine, 4-fluoro-phenylalanine, phenylalanine, 4-iodo-phenylalanine, 4-nitro-phenylalanine, phosphotyrosine, 4-tert-butyl-phenylalanine, 2-(trifluoromethyl)-phenylalanine, 3-(trifluoromethyl)-phenylalanine, 4-(trifluoromethyl)-phenylalanine, 3-amino-L-tyrosine, 3,5-diiodotyrosine, 3-amino-6-hydroxy-tyrosine, tyrosine, 3,5-difluoro-phenylalanine and/or 3-fluorotyrosine

In some embodiments, the amino acid derivative utilized in accordance with the invention comprises at least one DOPA group and at least one other amino acid selected from any amino acid defined herein. In some embodiments, the at least one other amino acid is a negatively charged amino acid. In some embodiments, the at least one other amino acid is a positively charged amino acid. In some embodiments, the at least one other amino acid is an aromatic amino acid. In some embodiments, the at least one other amino acid is a kinase-active or kinase-modifiable amino acid. It should be appreciated that the invention further encompass any of the peptides, any serogates thereof, any salt, base, ester or amide thereof, any enantiomer, stereoisomer or diasterioisomer thereof, or any combination or mixture thereof.

In some embodiments, the at least one other amino acid is selected from valine, isoleucine, leucine, methionine, phenylalanine, tryptophan, cysteine, alanine, tyrosine, histidine, threonine, serine, proline, glycine, arginine, lysine, arginine, aspartic acid, glutamic acid, asparagine and glutamine.

In some embodiments, the at least one other amino acid is selected from lysine, histidine and glutamic acid.

In some embodiments, the peptide is a dipeptide comprising DOPA and an amino acid selected from lysine, histidine and glutamic acid.

In some embodiments, the peptide comprises DOPA and two or more other amino acids, at least one of said two or more other amino acids is selected from Lysine, Histidine and Glutamic acid.

In some embodiments, DOPA-X is a peptide, wherein X is one or more amino acids connected to each other via a peptide bond or via a linker. The one or more amino acid may be selected as above. In some embodiments, X is selected from lysine, histidine and glutamic acid.

In some embodiments, in an amino acid derivatives of the form AA-X, used in accordance with the invention, AA may be a peptide and X may be selected as herein. The peptide may be a dipeptide, a tripeptide, a tetrapeptide or a higher homologue, such that variant X is one or more variant groups substituting any atom of the peptide or substituting any atom of any of the amino acid making up the peptide.

In some embodiments, X is Lysine, or X is Histidine, or X is Glutamic acid. In some embodiments, the at least one amino acid or a derivative thereof is selected to modify the environment inside the nanopore or at the vicinity of the nanopore. In some further embodiments, the at least one amino acid or a derivative thereof is selected to render the nanopore hydrophilic or hydrophobic.

In some embodiments, the amino acid derivative or peptide comprises DOPA and at least one amino acid selected to modify the environment inside the nanopore or at the vicinity of the nanopore, the at least one amino acid is selected based on its size, pKa, functional groups, polarity, etc.

In some embodiments, the peptide is selected to render the nanopore hydrophilic or hydrophobic.

Thus, in another aspect, there is provided a method of modifying at least one property of a nanopore environment, the method comprising associating at least one surface of said nanopore with at least one amino acid or a derivative thereof, wherein the amino acid or derivative thereof is selected to endow the nanopore environment with the at least one property selected from polarity, charge, hydrophobicity and hydrophilicity, and wherein the amino acid derivative is optionally a DOPA-containing molecule.

For certain applications, the amino acids or derivatives thereof utilized in accordance with the invention may be selected to enable coupling (conjugation), through any of the amino acid residues, to another amino acid or a peptide or agent that enters the nanopore and comes into contact with the amino acids or derivatives thereof. This provides the ability to use the amino acid-modified nanopore for detection of agents that flow through the nanopores or for solid state peptide synthesis of longer peptides based on the DOPA-based peptides.

In some embodiments, the peptides may be modified by enzymes, such as:
peptides modified by kinase: e.g., HDGF 160-174 peptide is phosphorylated by ERK2 kinase (Zhuravel, R., E. Amit, S. Elbaz, D. Rotem, Y.-J. Chen, A. Friedler, S. Yitzchaik, and D. Porath, Atomic force microscopy characterization of kinase-mediated phosphorylation of a peptide monolayer. Scientific reports, 2016. 6: p. 36793); and others as listed in for example in http://www.cbs.dtu.dk/databases/PhosphoBase/pbase2/; or
peptides that may be modified by acetyltransferase: e.g., P53 carboxy-terminal peptide acetylated by p300 acetyltransferase (Gu, W. and R. G. Roeder, Activation of p53 sequence-specific DNA binding by acetylation of the p53 C-terminal domain. Cell, 1997. 90(4): p. 595-606).

In some embodiments, the peptides are part of protein-protein or protein-DNA interaction sites, e.g., Bcl-2 103-120 and NFκB 303-313 peptides that bind to ASPP2 protein (Rotem-Bamberger, S., C. Katz, and A. Friedler, Regulation of ASPP2 interaction with p53 core domain by an intramolecular autoinhibitory mechanism. PLoS One, 2013. 8(3): p. e58470).

In some embodiments, the peptides are peptides that can change conformation through ligand binding: e.g., β-ESH peptide which conformation is changed by the organophosphate toxin paraoxon (Liebes-Peer, Y., H. Rapaport, and N. Ashkenasy, Amplification of single molecule translocation signal using β-strand peptide functionalized nanopores. ACS nano, 2014. 8(7): p. 6822-6832).

Further, to modify any one parameter of the nanopore, the amino acid derivative may be DOPA-based peptides extended at the N-terminus and/or through any other functional group present on the peptide to fine-tune the properties of the nanopore. As an example for such extension, the amino acid or derivative thereof, e.g., peptide, may be extended at the N-terminus thereof with identical or different amino acid residue(s), which may be naturally occurring or synthetic amino acid residue(s), for e.g., inducing a constrain on the peptide conformation or for inducing bulkiness at the nanopore.

The ability to modify the peptide when associated to the nanopore surface, enables construction of the peptide in situ. Generally speaking, a nanopore may be modified by a plurality of peptides, in a single step, by contacting a solution of the already-made peptides with the solid substrate or by flowing the already-made peptides, in solution, through the nanopores. Alternatively, instead of reacting the full pre-made peptide with a surface of the nanopore, the nanopore surface may be contacted with e.g., DOPA or another surface-associating amino acid, and subsequently with one or more other or same amino acids (or peptide) under conditions permitting covalent bonding between e.g., DOPA and the one or more amino acids. The conditions for carrying out the two-step or multistep process are similar to those utilized in solid-stated peptide synthesis.

In some embodiments, for endowing the nanopore with a particular property, it is sufficient to modify the nanopore, as detailed herein, with DOPA alone, namely with only the amino acid DOPA.

The amino-acid modified nanopores or the peptide-modified nanopores of the invention have been determined to exhibit different characteristics than the bare nanopores, free of the amino acid derivatives or peptides, and thus may be used in a variety of tailored applications. For example, nanopores should be hydrophilic for applications such as the methods disclosed herein that are based on constant flow of ions, in solution, through the nanopore. SiN-based nanopore and other kinds of nanopores do not have sufficient hydrophilicities under ambient conditions. These kinds of nanopores can be physically (e.g. by plasma) or chemically (e.g. by piranha solution, which is an extremely hazardous reagent) treated in order to gain sufficient hydrophilicity for the above applications. The hydrophilicity after such treatments is, however, normally short lived (less than 24 hours). Covering SiN-based nanopores with at least one amino acid or a derivative thereof or with peptides, e.g., DOPA-His, DOPA-Lys and DOPA-Glu, keeps the nanopores hydrophilic enough for at least several months under ambient conditions without necessitating further treatment.

Modifying nanopores with amino acids or derivatives thereof further allows tuning of the nanopores for specific applications. For example, nanopores' modification with charged amino acids or peptides can improve their ability to detect and analyze analytes with opposite charge, e.g. nanopores modified by positively charged amino acids or peptides such as DOPA-Lys or DOPA-His (under neutral pH conditions) can have improved capabilities (in terms of, e.g., analytes capture rate and translocation dwell time) in detecting and analyzing negatively charged analytes such as DNA.

Thus, in another aspect, the invention provides a method of improving stability of a nanopore, the method comprising associating with a surface region of the nanopore at least one amino acid or a derivative thereof, the association being between the at least one amino acid or derivative thereof and at least one of (i) an outer rim surface region of the nanopore, (ii) an inner-pore region of the nanopore, and/or (iii) a circumference surface of the nanopore rim, wherein the amino acid is optionally 3,4-dihydroxyphenylalanine (DOPA) and wherein the amino acid derivative is optionally a DOPA-containing molecule.

As used herein, the term “stability” refer to the long term stability of the amino acid-modified nanopore, i.e., shelf-life or usability period, or its thermal stability, resistance to oxidation, stability under acidic or basic conditions, etc. Thus, improving the stability of the amino acid-modified nanopore refers to an increased stability as defined above in comparison with an unmodified nanopore.

In some embodiments, the shelf-life may be increased by hours, days or months.

As shown in the Examples, a way to evaluate the stability of an amino acid-modified nanopore of the invention is to determine the variability in the currents through the amino acid-modified nanopore in comparison with an unmodified nanopore.

In some embodiments, the stability is improved by a decrease in the currents variability of 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95% or 100% in comparison with an unmodified nanopore.

The amino acid or peptide coating of the nanopore, as described herein, may be removed in order to be replaced by another. In some embodiments, the coating may be removed by cleaning the amino acid-modified nanopore surface with plasma and/or SDS. In some embodiments, the coating may be removed by heating the amino acid-modified nanopore surface to a temperature of about 200° C.

Nanopores can also be used for detecting changes in their surroundings. For instance, nanopores modified with amino acid derivatives or peptides, such as DOPA-His or DOPA-Glu, can be used to detect the pH of a solution that surrounds them, based on their currents at various pHs. As shown in the Examples, each of the peptides demonstrates sensitivity to a different pH range.

Amino acid-modified nanopores can be utilized for detecting and studying the activity of enzymes (such as kinases, phosphatases). Modification with relevant amino acid derivatives or peptides, that are affected by such enzymes, allows measurement of the activity of these enzymes either be detection of the enzymes binding to the amino acid derivatives or peptides in the pore and/or by detection of changing in the amino acid derivatives or peptides structure that is mediated by the enzyme activity (such as peptide phosphorylation by kinase). The detection is carried out by measuring a change in the electric currents in the systems. Detection and analysis of enzymes activities can be used for various applications, such as those in medicine and research, e.g., as exploring regulators of enzymes; measuring enzymes concentrations in body fluids and others.

Amino acid-modified nanopores can be utilized for detecting and studying interaction between proteins or interactions between a protein and DNA. Nanopores modified with amino acid derivatives or peptides, which can be made from fragments of one target protein, can be used to detect the binding of a second target protein. The detection may be made by measuring a change in the electric currents measured. These measurements can be used for various applications that are relevant for medicine and research, such as exploring regulators that affect these interactions; measuring protein concentrations in body fluids and others.

Amino acid derivatives or peptides can be used to link other molecules and macromolecules to the nanopore surface. Many chemical and physical interactions are known between amino acid derivatives or peptides and their residues (natural and un-naturals) and molecules. Linking of such molecules to nanopores through the amino acid derivatives or peptides can further expand the range of applications. The interaction between the amino acid derivatives or peptides and the molecules can be done either before or after binding to the nanopore. For example, to bind a DNA oligonucleotide modified with a thiol to a peptide that contains Cys residue; a nanopore modified with this combined molecule can be used, for instance, to study interactions between DNA and proteins.

The amino acid derivative or peptide modifying the nanopore may be associated with any surface region of the nanopore or at the vicinity of the nanopore. In some embodiments, the amino acid derivatives or peptides are associated with the nanopore rim, at either or both nanopore opening(s), the nanopore interior region at the vicinity of the rim(s), with a surface region within the nanopore channel, or with any region at the vicinity of the nanopore.

The nanopores may be part of a device, e.g., an electronic device. The electronic device may comprise a measuring unit.

Thus, in some embodiments, the present invention provides a device comprising (i) an amino acid-modified nanopore structure or assembly, as defined herein, and (ii) a measuring unit.

Methods according to the invention may be carried out when the device comprising the nanopore structure is constructed of two chambers comprising an electrode assembly constructed of a set of at least two electrodes. In some embodiments, each chamber is equipped with an electrode or an electrode assembly. In some embodiments, the electrode is an Ag/AgCl electrode.

When the chambers are filled with an electrolyte solution, flow of solution may be permitted through the nanopore from the first opening to the second opening via the interior of the nanopore. Thus, the two separate chambers are in liquid or gas communication.

In some embodiments, the device comprises a microfluidic system enabling changing sample solution. In some embodiments, the device comprises a cooling-heating system to control the temperature of the device. These systems and any additional system used in the device may be manually or controlled by a computer. In some yet other embodiments, and in order to reduce possible noise, the device may be placed within a Faraday cage and even on top of a vibration isolation table.

The device further comprises a measuring unit. The measuring unit is adapted to measure ionic current through the nanopore. In some embodiments, the ionic current is generated and measured by the same unit. In some other embodiments, different units are required to generate and measure the current. In some embodiments, the unit may be a voltage source, patch clamp system. In some embodiments, the generating and/or measuring unit may be further equipped with an amplifier and/or a low pass filter and/or digitizer.

In some embodiments, the measuring unit comprises a computer readable system.

The nanopore structure and the device comprising the structure may be used for a variety of applications.

One unique utility of a modified nanopore structure according to the invention is the ability to analyze a sample; when the sample is placed in close proximity to the nanopore or alternatively in the chamber and allowed to pass through the nanopore, the sensitivity and specificity of the nanopore structure allows monitoring translocation of analytes. When voltage is applied to the hybrid nanopore and no analyte is presented near the nanopore or in the chamber, a stable ionic current representing an open pore current may be measured. When an analyte is added near the nanopore or to the chamber near the nanopore, the analyte may pass through the nanopore to the other side of the nanopore. When the analyte is present near or inside the nanopore, part of the ionic flow in the nanopore is changed, causing a detectable change in ionic current. The change may be an increase in the current or blockade in current. This signal (transient) may be dependent on different parameters, for example the properties of the nanopore, electrolyte solution, and the passing molecule. Thus, the modified nanopore provides a fundamental tool for sample analysis.

Thus, in another aspect there is provided a method for analysis of at least one analyte in a sample comprising: (a) flowing a sample comprising at least one analyte or suspected to comprise at least one analyte through an amino acid-modified nanopore structure according to the invention; and (b) determining at least one of (i) presence or absence of an analyte in the sample, (ii) identity of the analyte in the sample, and (iii) concentration of the analyte in the sample, e.g., by monitoring at least one measurable parameter related to the nanopore indicative of the passing of an analyte through the nanopore.

In some embodiments, the method of analysis comprises: (a) applying a sample comprising at least one analyte or suspected to comprise at least one analyte onto an amino acid-modified nanopore structure according to the invention, (b) permitting the sample to flow through the nanopore; and (c) determining at least one of (i) presence or absence of an analyte in the sample, (ii) identity of the analyte in the sample, and (iii) concentration of the analyte in the sample, e.g., by monitoring at least one measurable parameter related to the nanopore indicative of the passing of an analyte through the nanopore.

The at least one measurable parameter may be a chemical parameter, or a physical parameter, or an optical parameter, or an electrical signal. Several measurable parameters may be obtained when the analyte is near or in the nanopore. In some embodiments, a change in the current (or the current value) may be detected. This change in the current may be determined (measured) by comparing an observed current to a current measured at an earlier time point, e.g., in the absence of a sample, and determining the ratio of the values between the two measurements. The change in the current may be either a blockage or an increase in the current. In some embodiments, a blockage (drop) in the current may be observed and, e.g., subsequently compared to a previous measurement.

In some embodiments, the change in current may be expressed as the fraction or percentage of the open nanopore current, open channel current, I/Io, where I is the blockade current and Io is the open channel current (e.g., in case an analyte is not detected). In some embodiments, the current blockade as noted above may indicate that an analyte is present at a region proximal to hybrid nanopore or in the nanopore structure, e.g., during passage through the hybrid nanopore channel.

In some embodiments, the change in current may be defined as an event having measurable time duration. The time duration of the change in the current or the time duration of a measurable or observed or detected event refers to the period over which the change in current occurs (measurable in millisecond, seconds, etc). In some embodiments, the measured time of the change (event) may reflect on the translocation time (passing) of a sample or an analyte, as defined herein, through the nanopore structure. The period over which the change in the current occurs may be determined as the time difference between a time point when a first current change (increase or blockage) is observed and a later time point when the change is arrested or further altered. In some embodiments, the time period is measured until a further change in the blockage or increase in the current is observed. This may be usually determined over a threshold value that is set beyond the baseline noise level. The time duration of the change may be fitted by Gaussian or by exponential with a time constant.

In some embodiments, the events are represented by transient spikes (indicative of one or more change in a measurable current). In some embodiments, the event integral, as described herein, may be determined by calculating the integral of ionic current over the duration of an event.

In some embodiments, the at least one measurable parameter is at least one of (i) change in current, and (ii) time duration of a change in the current and any combination thereof. In some embodiments, the at least one parameter may be determined manually by visual inspection or by automated means, including computational analysis, for example by application of appropriate algorithms.

A “sample” according to the present invention may be any sample including, but not limited to, biological samples obtained from biological systems (including cell cultures, micro-organism cultures), biological samples obtained from subjects (including humans and animals), samples obtained from the environment for example soil samples, water samples, agriculture samples (including plant and crop samples), food samples. The sample may also be body fluids such as whole blood, blood cells, bone marrow, lymph fluid, serum, plasma, urine, sputum, saliva, feces, semen, spinal fluid or CSF, external secretions of the skin, respiratory, intestinal, and genitourinary tracts, tears, milk, any human organ or tissue, any biopsy, for example, lymph node or spleen biopsies, any sample taken from any tissue or tissue extract, any sample obtained by lavage optionally of the breast ductal system, plural effusion, samples of in vitro or ex vivo cell culture and cell culture constituents.

In some embodiments, the sample is a liquid sample. In some embodiments, the liquid sample is a liquid in its natural state. In some further embodiments, the liquid sample is pre-treated to be in a liquid state. Pre-treatment may be by any method that changes a sample that is not a liquid in its natural state into a liquid state. In some embodiments, pre-treatment is by extraction. In some other embodiments, the sample comprises at least one liquid fraction.

The “analyte” which presence is to be determined or quantified is any molecule or ion which may be found in a sample. In some embodiments, the sample may comprise a binding agent capable of binding to the analyte prior to or during passing through the nanopore (or hybrid nanopore). The “binding agent” may be any molecule capable of specifically binding to the analyte, for example an aptamer, an antibody, a receptor ligand or a molecular imprinted polymer.

In some embodiments, the analyte may be a protein, a polypeptide, a peptide, a ganglioside, a lipid, a phospholipid, a carbohydrate, a small molecule or a nucleic acid. Non-limiting examples in accordance with the invention are soluble cancer markers, inflammation-associated markers, hormones, cytokines, drugs, and soluble molecules derived from a virus, a bacteria or a fungus for example, toxins or allergens. In some embodiments, the analyte is a cancer (or tumor) marker or a viral marker (or any fragment thereof). In general, a tumor marker may be found in the body fluids such as in blood or urine, or in body tissues. Tumor markers may be expressed or over expressed in cancer and are generally indicative of a particular disease process.

In some embodiments, the analyte is a nucleic acid.

In some embodiments, the analyte may be modified. In some embodiments, the analyte may be conjugated (chemically) to a moiety that may be any compound capable of producing a detectable signal. The moiety may be for example a chromophore, a fluorophore or a luminanophore. In some other embodiments, the at least one measurable parameter may be an optical signal. As appreciated, Alkaline Phosphatase (AP) or Horse Radish Peroxidase (HRP) substrate detection may be achieved by chromatic signal, fluorescence signal or luminescence signal, which may be detected using various spectrophotometers and fluorometers.

As used herein, the term “nucleic acid”, “nucleic acid sequence”, or “nucleic acid molecule” refers to polymers of nucleotides, and includes but is not limited to deoxyribonucleic acid (DNA), ribonucleic acid (RNA), DNA/RNA hybrids including polynucleotide chains of regularly and/or irregularly alternating deoxyribosyl moieties and ribosyl moieties, and modifications of these kinds of polynucleotides, wherein the attachment of various entities or moieties to the nucleotide units at any position are included. The terms should also be understood to include, as equivalents, analogs of either RNA or DNA made from nucleotide analogs, and, as applicable to the embodiment being described, single-stranded (such as sense or antisense) and double-stranded polynucleotides. In accordance with the present invention and as disclosed herein below, the analyte may be a nucleic acid molecule and in some embodiments of the present disclosure a modified nucleic acid molecule.

The invention further provides a method for sequencing a nucleic acid molecule comprising (a) applying a sample comprising at least one nucleic acid molecule onto a modified nanopore structure according to the invention, and determining the sequence of the nucleic acid molecule.

In some embodiments, the nucleic acid is DNA. In some other embodiments, the nucleic acid is RNA. In some embodiments, the nucleic acid is a double stranded (ds) nucleic acid. In some other embodiments, the nucleic acid is a single stranded (ss) nucleic acid.

When referring to sequencing of at least one nucleic acid molecule, it should be noted that the molecule may be a ds-DNA, ss-DNA, ds-RNA or ss-RNA. The nucleic acid may be a synthetic molecule or alternatively a nucleic acid molecule obtained from any biological sample, food sample or the like as described herein. In some embodiments, the nucleic acid subjected to analysis is in a linear conformation. In some further embodiments, the nucleic acid is an unstructured nucleic acid.

In another aspect, the present invention provides a method for the diagnosis of a condition in a subject comprising using an analysis method in accordance with the invention as described above. In some embodiments, the analyte is an analyte associated with the condition and wherein the presence or absence of analyte is indicative of the presence of a condition in the subject.

In another aspect, the present invention provides a method for monitoring the efficiency of a therapeutic regimen in a subject suffering from a condition comprising using an analysis method in accordance with the invention as described above. In some embodiments, the analyte is associated with the condition and wherein the amount of analyte is indicative of the level of the condition and thereby of the efficiency of the therapeutic regimen in the subject.

The invention further provides an amino acid-modified nanopore structure and/or device comprising same for use in research purposes. Non-limiting examples include laboratory use, scientific experiments and the like.

In further aspect, there is provided an amino acid-modified nanopore structure and/or device comprising same for use in analysis of at least one analyte in a sample. In some embodiments, the amino acid-modified structure is used in determining at least one of (i) presence or absence of an analyte in the sample, (ii) identity of the analyte in the sample, (iii) concentration of the analyte in the sample.

In accordance with the present disclosure, the amino acid-modified structure is used in sequencing a nucleic acid molecule.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 presents non-limiting examples of amino acid modified molecules, e.g., dipeptides, used in accordance with the invention: DOPA-His (1), DOPA-Lys (2) and DOPA-Glu (3) structure.

FIGS. 2A-2B: in FIG. 2A the currents shown at 100 mV through 12 nm nanopores, one treated with DOPA-His dipeptides and one that was unmodified; the measurements were repeatedly for 5 times in the same day at 1 M KCl, 10 mM tris-HCl, 1 mM EDTA (pH 7.5). FIG. 2B shows current stability of the same pore (DOPA-His treated) over several months, under the same conditions.

FIGS. 3A-3B: in FIG. 3A conductance measurements are shown through di-peptides coated nanopores as a function of pH change. Measurements were performed in 0.14 M KCl, 10 mM tris-HCl/succinic acid, 1 mM EDTA (pH 4.5, 6, 7.5 and 9) at 100 mV. FIG. 3B shows conductance measurements through di-peptides coated nanopores as a function of pH change. Measurements were performed in 1 M KCl, 10 mM tris-HCl/succinic acid, 1 mM EDTA (pH 4.5, 6, 7.5 and 9) at 100 mV.

FIGS. 4A-4C: in FIG. 4A 2 Kbp DNA translocation is demonstrated through DOPA-His modified nanopore, by applying 400 mV through the membrane. FIG. 4B shows a dwell time histogram for 2 Kbp DNA translocation through DOPA-His coated (thin line) and uncoated Si₃N₄ pore (bold line). The measurement was done at 1 M KCl, 10 mM Tris-HCl, 1 mM EDTA, and pH 7.5, 200 mV. FIG. 4C shows a dwell time histogram for 48 Kbp DNA, through DOPA-His coated (thin line) and uncoated Si3N4 pore (bold line).

FIGS. 5A-5C show translocation recording for 2 Kbp DNA using 12 nm pores coated with DOPA-His at 1 M KCl, 10 mM tris-HCl, 1 mM EDTA, 10% glycerol at pH 7.5, 200 mV. FIG. 5A shows current recordings showing blockade events at different DNA concentrations. FIG. 5B shows a plot of the rate as a function of DNA concentration, showing linear dependence. FIG. 5C shows a scatter plot for amplitude (pA) vs. dwell time (ms).

FIGS. 6A-6D show translocation recording for 2 Kbp DNA using 10 nm pores coated with DOPA-His at 1 M KCl, 10 mM tris-HCl, 1 mM EDTA, 10% glycerol at pH 7.5, at 100, 200 and 300 mV. FIG. 6A shows a scatter plot of dwell time (ms) and amplitude (pA). FIG. 6B shows a histogram, showing dwell time distribution as a function of three voltages. FIG. 6C shows the exponential dependence of dwell time as a function of voltage. FIG. 6D shows exponential dependence of events frequency as a function of voltage.

### DETAILED DESCRIPTION OF EMBODIMENTS

### Non-Limiting Examples

Methods

Nanopore Fabrication

Nanopores were fabricated in 30 nm thick, low-stress SiN windows (50×50 μm²) supported by a silicon chip (Protochips) using a focused electron beam of a 200 keV TEM (Tecnai, F20 G2). Once the pores were drilled, they were stored in ethanol:ddH2O (1:1, v:v) immediately to avoid any contamination.

Exemplary Protocol for Preparation of a Peptide According to the Invention: Di-Peptide Preparation

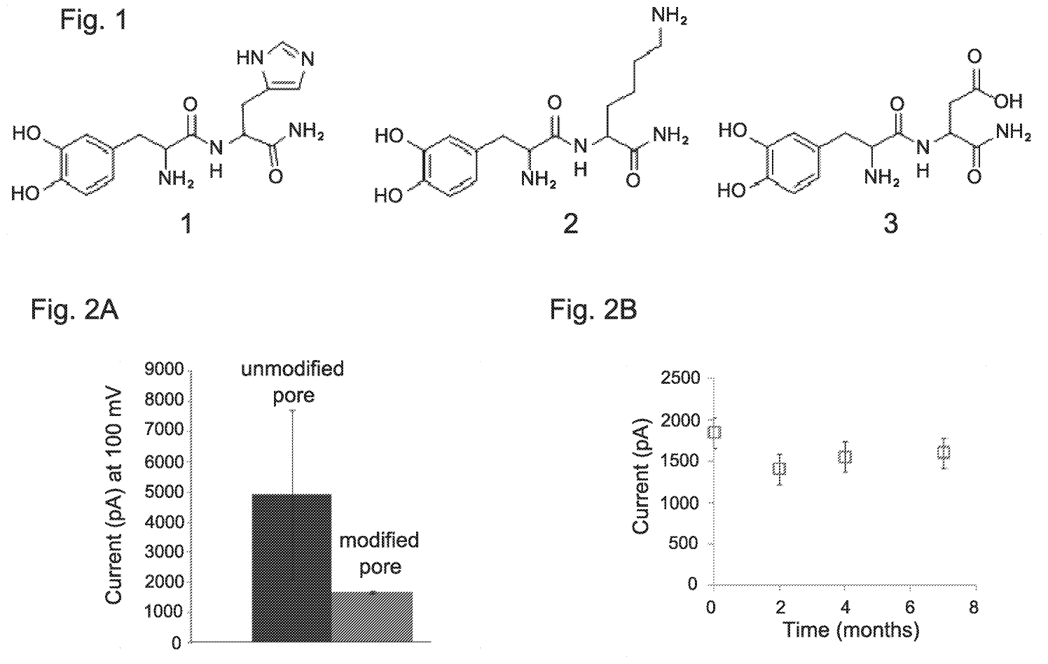

The dipeptides DOPA-His, DOPA-Lys and DOPA-Glu (FIG. 1) were synthesized using 9-fluorenylmethoxycarbonyl (Fmoc) based solid-phase peptide chemistry manually. Standard coupling conditions using AA/HATU/DIPEA were employed to obtain the desired peptides. The peptides were synthesized on Fmoc-Rink amide resin which was subjected to Fmoc removal before coupling the AA residues to yield C-terminus amides. Amino acids were coupled in 5 fold excess in the synthesis and all residues were coupled once for 1 h. The coupling reactions were monitored by Kaiser ninhydrin test. Removal of the Fmoc group was performed using 20% Piperidine in DMF for 15 min for two times and the residual piperidine was removed by three consecutive washes with DMF. Peptide cleavage from the resin support was performed using 95% trifluoroacetic acid, 2.5% water and 2.5% triisoproplysilane (5 mL/183 mg of resin) for 2 h at room temperature, followed by precipitation in cold diethyl ether. The precipitated peptide was redissolved in 50% acetonitrile and lyophilized to obtain crude peptide as white solids. The crude peptides were purified by preparative high performance liquid chromatography. Peptide identity was confirmed using electrospray ionization mass spectrometry (ESI-MS, Waters ZQ4000, Waters Corp., Woburn, Mass.). Pure peptides were stored at −20°.

Nanopore Modification with Di-Peptide

Nanopore membrane were treated in a Plasma Cleaner for 30 see before modification with di-peptide to improve binding. Nanopore membrane was immersed in di-peptide solution (0.5 mg/ml di-peptide dissolved in tris-HCl:ethanol (1:1, v:v)) for overnight at room temperature and then washed with 3 ml ethanol.

Nanopore Recording

Coated chip including the pore is mounted in in a custom electrophoresis flow cell. Two reservoirs on each side with a volume of 100 μl (trans and cis) were filled with filtered and degassed buffer of 140 mM KCl, 10 nM tris-HCl, 1 nM EDTA at various pH values (4.5, 6, 9). A pair of Ag/AgCl pellet electrodes was immersed in the two reservoirs and connected to an Axopatch 200B amplifier (Molecular Devices, Inc.) to record ionic current flow through the nanopore. The whole setup was put in a double Faraday cage to lower external electrostatic interference. Signals were collected at 10 kHz sampling rate using a Digidata 1440A (Molecular Devices, Inc.) and filtered at 1 kHz using the built-in low pass Bessel filter of Axopatch.

### Example 1

Peptide Modification Increases the Nanopore Usability

The nanopore are optionally hydrophilic for application where ions and charged ligands are required to pass through them. The nanopores that were treated in a Plasma Cleaner for 30 sec before recording were compared to ones that were treated with DOPA-His dipeptides. The currents through the nanopores were measured repeatedly for 5 times in the same day. After each measurement, the chips were washed with water to remove the salt residues, and then installed back in the flow cell. During the repeated measurements, the variability in the currents through the peptide-modified nanopore was much smaller in comparison with the one in the unmodified nanopore (FIG. 2A). In addition, similar currents were measured through peptide-coated nanopore for at least months, without any additional treatment (FIG. 2B). Unmodified nanopores, on the other hand, can conduct current only for a few hours after treatment with plasma or piranha. These results indicate that the peptide coating improves the nanopore stability and usability both for the short and long terms.

### Example 2

pH Effect on the Peptide Modified Nanopores Conductance

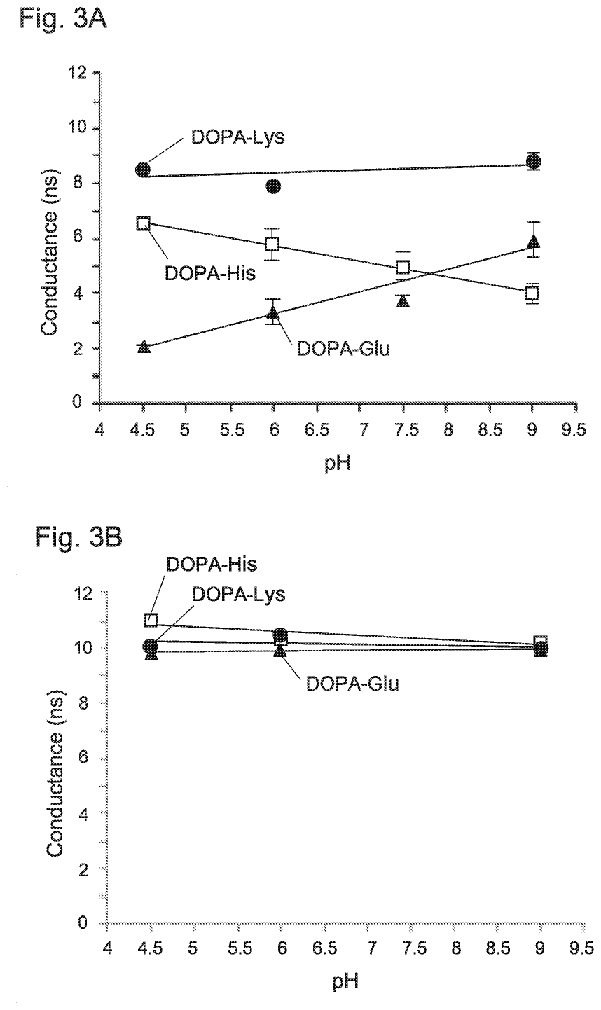

The conductivity of each di-peptide modified nanopore were measured at pH 4.5, 6, 7.5 and 9. At 0.14 M KCl solution, the conductivity of DOPA-His modified nanopore was reduced as the pH increases, the conductivity of DOPA-Glu modified nanopore was increased as the pH increases and no effect was observed at DOPA-Lys nanopore (FIG. 3A). These results are in good correlation with the pKa values of the side chains of Histidine (6.04), Glutamic acid (4.25) and Lysine (10.79) that affect the charge and therefore the hydrophilicity of these amino acids. When the salt concentration was increased to 1 M KCl, all three pores exhibited a weak pH dependence on conductance, if at all (FIG. 3B). The results suggests that peptide coated nanopores can thus be utilized as small and sensitive pH and salt concentration sensors and can be adjusted based on the coated peptide identity.

### Example 3

DNA Translocation Through Di-Peptide Modified Nanopores

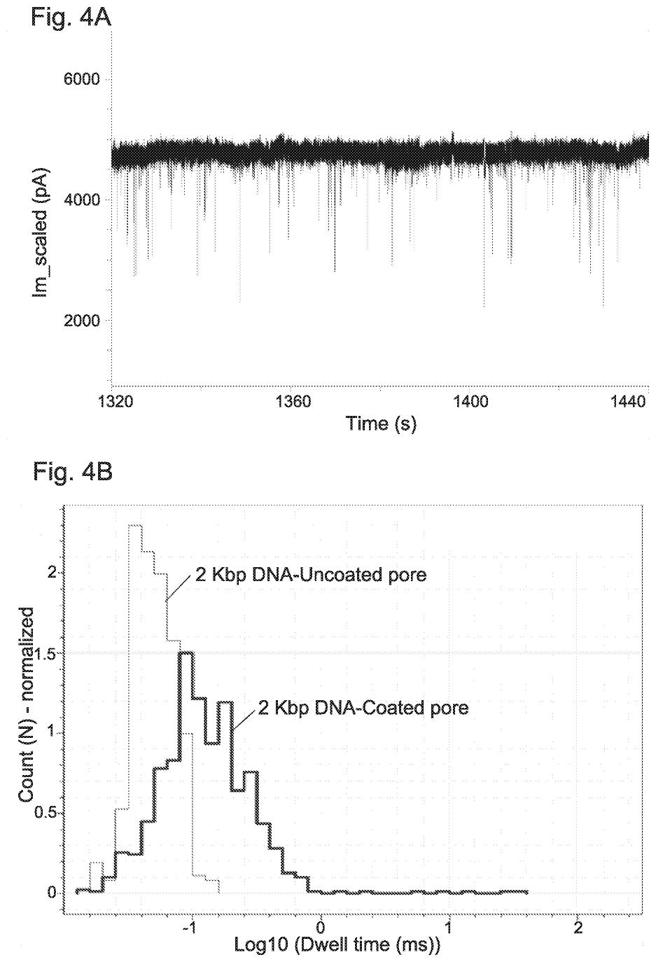

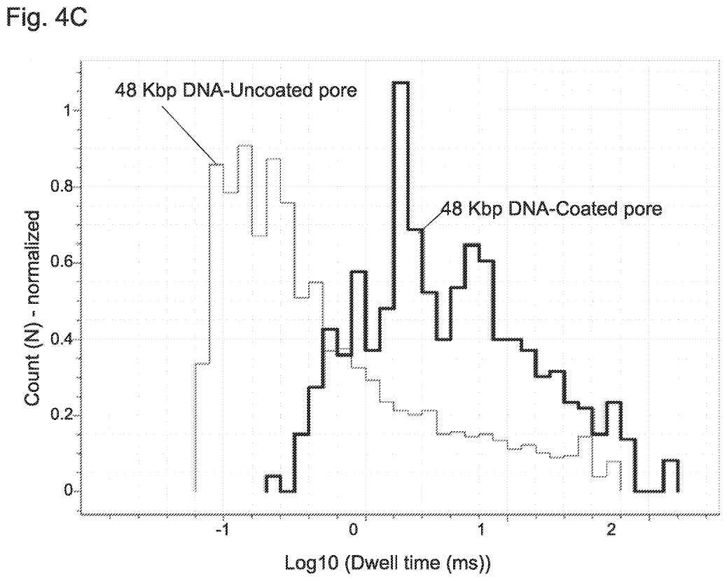

To demonstrate the ability of the peptide-modified nanopores to detect analytes, the translocation of 2 Kbp DNA through the peptide modified nanopores was measured. Blockage events were observed both in DOPA-His (FIG. 4A) and DOPA-Lys nanopores that are positively charged at the measurement pH (7.5), but not in DOPA-Glu nanopore that is negatively charged at the same pH. The DOPA-His coating reduce the DNA translocation in about order of magnitude compare to uncoated nanopore (FIG. 4B-4C). The slowing down may result from either the positive charge residing in the coated pore (His is positively charged at pH 7.5) and/or from a possible higher friction. The translocation of a longer fragment of DNA i.e. 48 Kbp was also tested using DOPA-his coated nanopore (FIG. 4C). As expected, the translocation of the longer DNA fragment (48 Kbp) has longer dwell time, 5 ms than the short one (2 Kbp), 0.1 ms (FIG. 4B).

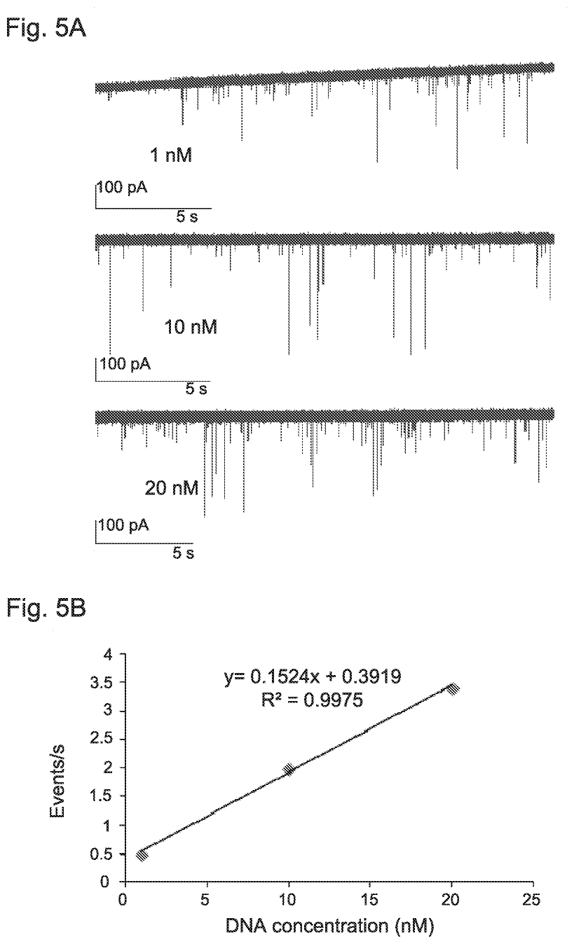

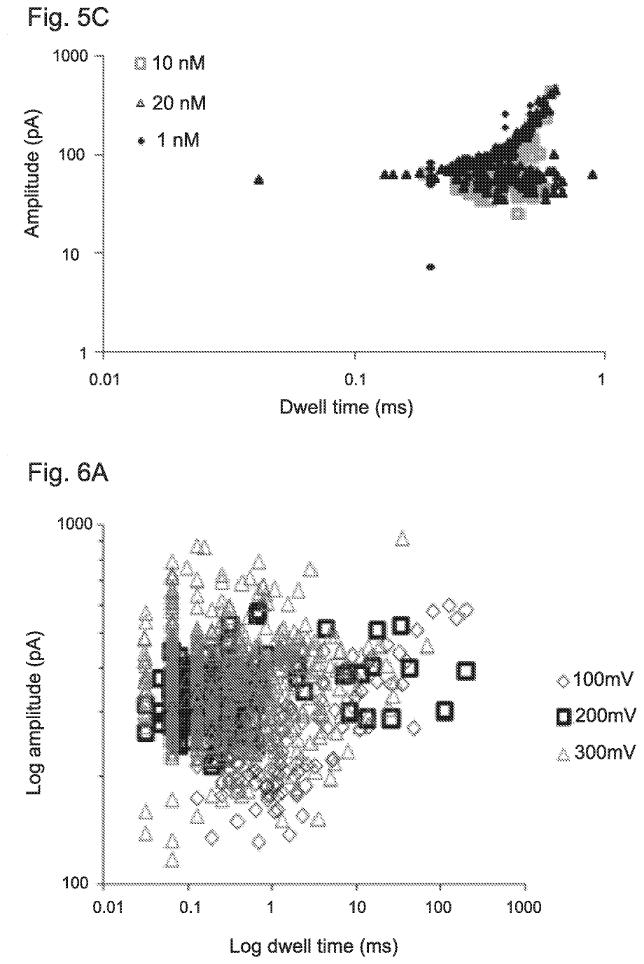

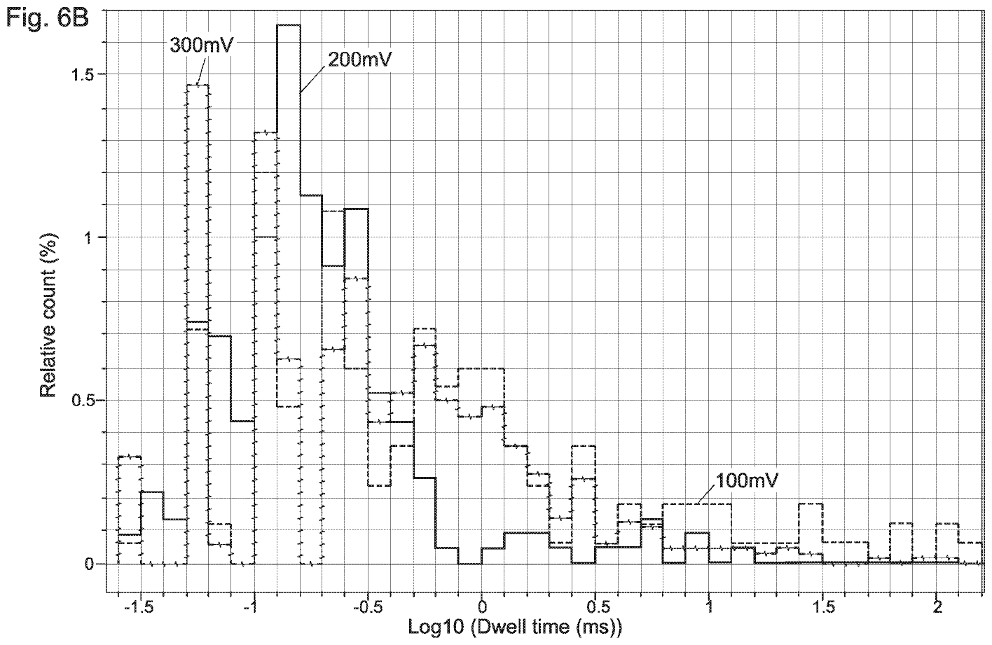

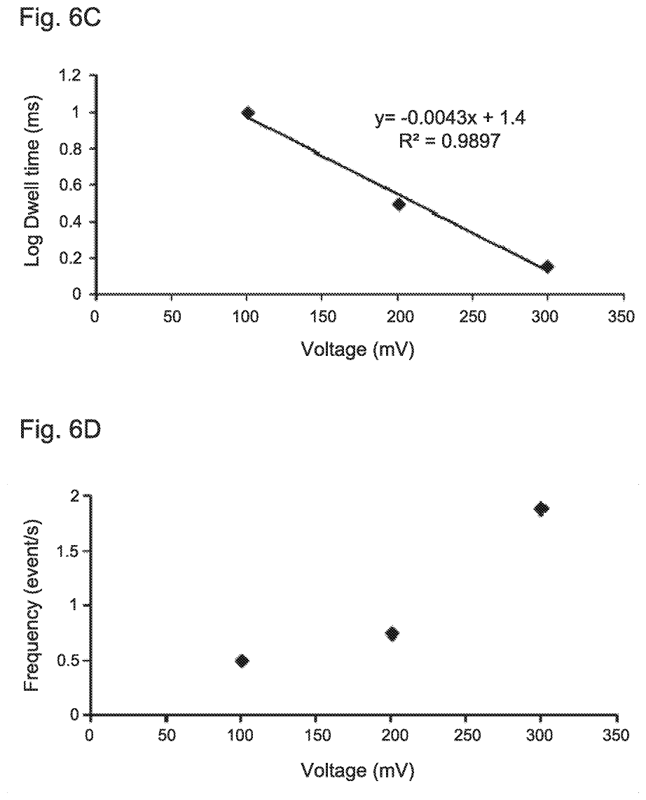

Several concentrations of the small DNA fragment (2 Kbp) were also tested using the DOPA-his coated nanopore (FIG. 5A). It was observed that the rate of blockade events correlates with the DNA concentration (FIG. 5B-5C). Since electrophoretic dragging of the DNA through the pore is the kinetic driving force, an exponential dependence of dwell time on the voltage was expected. FIG. 6A shows the dwell time and amplitude distribution of DNA translocation measured at various driving voltages for nanopore coated with DOPA-His. The distributions of dwell time for DNA translocation under different potentials were plotted in FIG. 6B. An exponential dependence of the dwell time (FIG. 6C) on the voltage was observed, which is in good agreement with electrophoretic-force driven translocation. An exponential dependence of the frequency of the events on the voltage was also observed (FIG. 6D). These results confirm that dsDNA was translocated through the coated nanopore.

1. A nanopore surface-associated with at least one amino acid or a derivative thereof, the association being between the at least one amino acid or derivative thereof and at least one of (i) an outer rim surface region of the nanopore, (ii) an inner-pore region of the nanopore, and/or (iii) a circumference surface of the nanopore rim, wherein the amino acid is optionally 3,4-dihydroxyphenylalanine (DOPA) and wherein the amino acid derivative is optionally a DOPA-containing molecule.

2. The nanopore according to claim 1, wherein the amino acid derivative is of the form AA-X, wherein AA is the amino acid, X is one or more variant groups covalently associated with DOPA, and “-” designates one or more covalent bonds.

3. The nanopore according to claim 1, wherein the amino acid is DOPA and the amino acid derivative is DOPA-X, wherein X is a variant group covalently associated with DOPA.

4-5. (canceled)

6. The nanopore according to claim 1, wherein the amino acid is selected on the basis of their polarity, charge, solubility, hydrophobicity, hydrophilicity, and/or the amphiphatic nature.

7. The nanopore according to claim 1, wherein the amino acid is selected from valine, isoleucine, leucine, methionine, phenylalanine, tryptophan, cysteine, alanine, tyrosine, histidine, threonine, serine, proline, glycine, arginine, lysine, arginine, aspartic acid, glutamic acid, asparagine and glutamine.

8. The nanopore according to claim 1, wherein the amino acid is selected from lysine, histidine and glutamic acid.

9. The nanopore according to claim 2, wherein X is selected from hydrophobic groups, hydrophilic groups, electron withdrawing groups, electron donating groups, bulky groups, single atom substituents, and binary substituents.

10. (canceled)

11. The nanopore according to claim 2, wherein X is an amino acid and DOPA-X is a peptide.

12. The nanopore according to claim 11, wherein the peptide is a dipeptide comprising DOPA and an amino acid selected from lysine, histidine and glutamic acid.

13. The nanopore according to claim 11, wherein the peptide comprises DOPA and one or more other amino acids, at least one of said one or more other amino acids is selected from lysine, histidine and glutamic acid.

14. (canceled)

15. The nanopore according to claim 11, wherein X is lysine, or X is histidine, or X is glutamic acid.

16. The nanopore according to claim 1, wherein the amino acid or derivative thereof is selected to modify the environment inside the nanopore or at the vicinity of the nanopore.

17. The nanopore according to claim 16, wherein the amino acid or derivative thereof is selected to render the nanopore hydrophilic or hydrophobic.

18. The nanopore according to claim 2, wherein the peptide comprises DOPA and at least one amino acid selected to modify the environment inside the nanopore or at the vicinity of the nanopore.

19. The nanopore according to claim 18, wherein the peptide is selected to render the nanopore hydrophilic or hydrophobic.

20. A device comprising (i) a structure comprising a plurality of nanopores according to claim 1, and (ii) a measuring unit.

21. A method of improving stability of a nanopore comprising associating at least a surface region of the nanopore with at least one amino acid or a derivative thereof, the association being between the at least one amino acid or derivative thereof and at least one of (i) an outer rim surface region of the nanopore, (ii) an inner-pore region of the nanopore, and/or (iii) a circumference surface of the nanopore rim, wherein the amino acid is optionally 3,4-dihydroxyphenylalanine (DOPA) and wherein the amino acid derivative is optionally a DOPA-containing molecule.

22. (canceled)

23. A method of modifying at least one property of a nanopore environment, the method comprising associating at least one surface of the nanopore with at least one amino acid or a derivative thereof, wherein the amino acid is selected to endow the nanopore environment with the at least one property selected from polarity, charge, hydrophobicity and hydrophilicity, and wherein the amino acid derivative is optionally a DOPA-containing molecule.

24. A method for analyzing at least one analyte in a sample, the method comprising: (a) flowing a sample comprising at least one analyte or suspected to comprise at least one analyte through an amino-acid modified nanopore according to claim 1; and (b) determining at least one of (i) presence or absence of an analyte in the sample, (ii) identity of the analyte in the sample, and (iii) concentration of the analyte in the sample.

25. The method according to claim 24, wherein said analyte is any one of a nucleic acid molecule, a protein, a polypeptide, a peptide, a ganglioside, a lipid, a phospholipid, a carbohydrate or a small molecule.
